(19) United States
(12) Patent Application Publication
(10) Pub. No.: US 2004/0096815 A1
(43) Pub. Date: May 20, 2004

(54) Method of identifying an eventual modification of at least one biological parameter making use of living cells which are subjected to a stress and living cells which are not subjected to this same stress

(75) Inventors: Eric Perrier, Les Cotes d'Arey (FR); Valerie Andre, Ampuis (FR); Stephane Grenier, Luzinay (FR); Corinne Reymermier, Charly (FR)

Correspondence Address: MERCHANT & GOULD PC, P.O. BOX 2903, MINNEAPOLIS, MN, 55402-0903, US

(73) Assignee: COLETICA, Lyon (FR)

(21) Appl. No.: 10/365,853

(22) Filed: Feb. 12, 2003

(30) Foreign Application Priority Data
Nov. 19, 2002 (FR) 02 14491

(51) Int. Cl. C12Q 1/02; C12Q 1/00

(52) U.S. Cl. 435/4

(57) ABSTRACT

An aim of the invention is a method of identifying an eventual modification of at least one biological parameter.

The present invention relates essentially to a method of identifying an eventual modification of at least one biological parameter, characterized in that it comprises the compared proteomic and/or compared transcriptomic and/or compared genomic analysis:

a) of living cells which are subjected to a stress, called stressed cells,

b) of living cells which are not subjected to this same stress, called reference cells,

c) at least one of these two classes of cells being used in a three-dimensional tissue model,

enabling eventually identifying at least one biological parameter which is modified following said stress. The invention comprises the use of this process for the screening of active principles.

The present invention relates essentially to a method of identifying an eventual modification of at least one biological parameter, comprising the compared proteomic and/or compared transcriptomic and/or compared genomic analysis:

a) of living cells which are subjected to a stress, called stressed cells,

b) of living cells which are not subjected to this same stress, called reference cells,

c) at least one of these two classes of cells being used in a three-dimensional tissue model,

enabling eventually identifying at least one biological parameter which is modified following said stress.

The present invention further relates to a method of identifying at least one potentially active substance capable of reversing at least one biological parameter which is modified which is during a stress, or to provide an indication of the modification of at least one biological parameter which is modified during a stress.

The present invention further relates to the use of an active substance selected by such a method, for preparing at least one cosmetic and/or pharmaceutical composition.

The present invention further relates to a substance which is active in the field of cosmetics or of pharmacy and which is selected by such a method.

### STATE OF THE ART

Solar radiation is formed from electromagnetic radiations including 56% of infrared radiation (wavelength of 5,000 to 800 nm) which generate heat, 39% of visible light (from 800 to 400 nm) and 5% of ultraviolets A, B, C (from 400 to 190 nm), including about 4.9% of UVA and 0.1% of UVB+UVC.

UVCs, which are very harmful, are in general filtered by the ozone layer.

UVBs are partially stopped in passing through the atmosphere and glass. They meet the epidermis, but are stopped before the dermis. They are responsible for the formation of a sunburn which is characterised by the presence of sunburn cells, which are keratinocytes which have started an apoptosis process due to lesions of the DNA of their nucleus. Their number is as high as the dose of UVB received is high. In fact, a natural process of defense enables repairing or removing harmed cells, however, the failure of this system by saturation or genetic fault plays a fundamental role in the appearance of skin cancers.

UVAs pass through the atmosphere easily, in Summer as in Winter, they penetrate the dermis and the epidermis and, although less harmful than UVBs, they are nonetheless responsible for damages due to the amounts received. UVAs do not produce, or produce very little, sunburn cells and they can damage cell DNA, but also cell lipids and cell proteins, via the formation of free radicals. UVAs are responsible for an accelerated skin ageing with an increased degradation of the collagen and elastic fibers, but also of the other constituents of the extra-cellular matrix within the dermis. Furthermore, many pieces of work have shown the immunosuppressent effects of UVAs via the epidermal Langerhans cells which, after irradiation, interact with the T lymphocytes and have a systemic effect through the cytokines and the neuromediators produced in a cascade by the epidermal cells and the nerve fibres (Meunier L., Eur. J. Dermatol. 1999, 9: 269-275). In the long term, the risk of skin cancer is increased, the pre-cancerous cells being no longer recognized as foreign cells and therefore no longer eliminated by the immune system.

A European survey Helios (Zanetti R. et al, Br. J. Canc. 1996, 73: 1440-1454) shows the relationships between exposure to UVs, skin phenotype and carcinomas, and defines the notion of direct risk linked to the UVs.

Amongst the other damages which are known and which are linked to the radiations, those may be cited which are due to an increase in the temperature by the infra-reds, which induce, on the melanocytes, the production of heat shock proteins, as well as effects which are similar to the induced UVB effects (Nakazawa et al, J Invest Dermatol 1998, 110: 972-977); those due to the near-infrared radiations affect the cell viability of the skin cells (YOO B. H. et al, J Cosmet Sci 2002, 53(3): 175-184); those due to visible light exposures (repeated doses) which induce a mutagenicity (Larko O., Lakartidningen 2002, 99(18): 2036-2040); those due to the ionizing radiations generate ulcerations and cancers (Lorette G., Machet L., Cancer Radiother 2001, 5(1): 116s-120s) or modifications of cell metabolism such as the synthesis of collagen of type I and III in the skin (Riekki R et al, Arch Dermatol Res 2002, 294(4): 178-184) or even modifications of the proliferation and of the epidermal differentiation (Sivan V. et al, Int J Radiat Oncol Biol Phys 2002, 53(2): 385-393), but also those due to the cytogenetic effects of microwaves (Zotti-Martelli L et al, Mutat Res 2000. 472(1-2): 51-58) or to the oncogenic effects by inhibition of the Heat Shock Proteins (HSP70 and HSP27) pathway, or by generation of free radicals by electromagnetic fields and notably those generated by mobile telephones (French P. W et al, Differenciation 2001, 67(4-5): 93-97; Di Carlo et al, J Cell Biochem 2002; 84(3): 447-454; Leszczynski D. et al , Differenciation 2002 70(2-3): 120-129; Moustafa Y. M. et al, J Pharm Biomed Anal 2001, 26(4): 605-608).

Other factors also exist such as certain chemical agents (hydrogen peroxide, nitric oxide, heavy metals, . . . ), or biological agents (viruses, bacteria, . . . ), even mechanical factors (stretching, compression . . . ) which can induce a cell stress.

At the present time, all the cell functions, including proliferation, differentiation and cell death, which are controlled by numerous genes and cell signal pathways, are analyzed ex vivo (biopsy) or in vitro on cell cultures in monolayer, generally of fibroblasts, keratinocytes or melanocytes originating from healthy or pathological donors or from cell-lines. Moreover, it is not always possible to find data on the expression and the cell syntheses as a function of a particular stress, or the results obtained in vitro sometimes emerge contradictory to what can be observed in vivo due to the simplified model used in the experimentation.

For several years, various three-dimensional cell models have been developed mainly for cell therapy, for cytotoxicity tests and effectiveness tests, alternative to experiments on animals. Models of epidermis can be cited (EP 0 789 074 A1 of Oreal; A. de Brugerolle de Fraissinette et al. 1999, Cell Biol. Tox. 15 : 121-135) used for the predictability of acute or chronic skin irritation, but also models of reconstructed epithelia (Schmalz G. et al., Eur. J. Oral Sci. 2000. 108 : 442-448; Nielsen et al., Int. J. Pharm. 2000. 200 : 261-270), as well as of reconstructed skins or of pigmented reconstructed skins and/or immunocompetent reconstructed skins for example (Regnier M. et al in Pour la science (1999) 266: 154-159).

If some of these models are today very widely used for the pharmaco-toxicological evaluations and the studies of effectiveness of pharmaceutical and cosmetic ingredients, the methods of analysis used are essentially techniques of histology combined with image analysis, analysis of metabolic syntheses and of their regulation by electrophoretic analysis, Western-blot Northern-blot or RT-PCR analysis. The techniques of protein array (or MAPPing) and DNA arrays, of bi-dimensional electrophoresis or of combined cytokine determinations (cytokine-MAP) in particular have not been applied to these three-dimensional models which are cultivated under standard culture conditions, in comparison with culture conditions under which these models have undergone a stress, whether it be of physical, chemical, biological or mechanical nature.

In contrast, these technologies have been developed and used on cell systems in monolayer such as, for example, in the study of the modulating role of an ultraviolet stress on normal or malignant human melanocytes (cell-line or melanocytes extracted from tumor tissue) cultivated in monolayer (Valery C., Grob J. J. and Verrando P. in J. Invest. Dermatol. (2001)117: 1471-1482).

### AIMS OF THE INVENTION

A main aim of the invention is to unexpectedly solve the technical problem which consists in providing a study model of cell metabolism, which reflects the situation observed in vivo, when the cells have undergone a stress.

An aim of the present invention is to solve the novel technical problem which consists in providing a method of identifying an eventual modification of at least one biological parameter, comprising the compared proteomic and/or compared transcriptomic and/or compared genomic analysis:

a) of living cells which are subjected to a stress, called stressed cells,

b) of living cells which are not subjected to this same stress, called reference cells,

c) at least one of these two classes of cells being used in a three-dimensional tissue model,

enabling eventually identifying at least one biological parameter which is modified following said stress.

This proteomic or transcriptomic or genomic analysis enables defining action targets so as to reverse or to provide an indication of the modification of at least one parameter which is modified during the stress applied.

Another aim of the present invention is to provide a solution which enables the use of three-dimensional tissue models described above with the view to evaluating the effect on the genomic or proteic profile of an active principle, in particular a cosmetic or pharmaceutical active principle.

Another aim of the present invention is to provide a solution which enables the use of three-dimensional tissue models described above with the view to evaluating the effect upon the genomic or transcriptomic or proteomic profile of a formulation, in particular a cosmetic or pharmaceutical formulation containing such an active principle.

Yet another aim of the present invention is to solve the novel technical problem consisting of providing a method of identifying at least one potentially active substance capable of reversing or of providing an indication of the modification of at least one biological parameter which is modified during a stress.

Yet another aim of the present invention is to solve the novel technical problem consisting of providing an active substance selected by such a method and its use for preparing a cosmetic or pharmaceutical composition.

### SUMMARY OF THE INVENTION

The present invention enables solving the technical problems set forth above.

Within the context of this invention, by <<genomic study>>, the inventors mean the act of drawing up an inventory of at least a part of the different genes which expressions are modified, in order to modify their expression.

By <<transcriptomic study>>, the inventors mean the act of drawing up an inventory of at least a part of the different RNAs which expressions are modified, in order to modify their expression.

By <<proteomic study>>, the inventors mean the act of drawing up an inventory of at least a part of the different proteins which expressions are modified, in order to modify their expression.

The invention consists mainly of providing a method of identifying an eventual modification of at least one biological parameter, characterized in that it comprises the compared proteomic and/or compared transcriptomic and/or compared genomic analysis

a) of living cells which are subjected to a stress, called stressed cells,

b) of living cells which are not subjected to this same stress, called reference cells,

c) at least one of these two classes of cells being used in a three-dimensional tissue model, enabling eventually identifying at least one biological parameter which is modified following said stress.

Cells which are called <<reference cells>> are cells which have not undergone the stress studied. It will be easily understood by the person skilled in the art that in order to optimize the invention described, the reference cells are cells which are the least possible exposed to whatever stress. These cells will notably be either removed on protected biopsies, i.e. which are not very much exposed to sun radiation (breast, abdomen, etc. . . . ), or cells which are not exposed to any stress whatsoever (physico-chemical, biological or mechanical stress).

Cells which are called <<stressed>> cells are cells which originate from biopsies taken in areas which are exposed to the sun (hand, face, etc. . . . ), or cells which are exposed to a stress (physical, chemical or biological stress), including the stresses: UVA, UVB, sunlight, infra-red, near infra-red, thermal, magnetic field, hertzian radiation including microwaves and waves of mobile telephones, ionizing radiation including beta, gamma, and X rays, such as those undergone during an accidental or non-accidental exposure to such radiation, etc. . . . . The cells can originate from several biopsies, from the same donor or from different donors.

The biological parameter corresponds in general to any modification of the expression of a gene, to any modification of the secretion of a protein, or to any modification which could be noted in the metabolism of a reference cell.

The tissue model is defined as being a tissue model, also called a three-dimensional model, which can be sown with living cells, notably with the aim of reconstituting a tissue of a living being, in particular the tissue model is defined as being able to be a model of connective matrix, called dermis in the case of skin and called chorion in the case of a mucous membrane, containing mainly stromal cells, an epithelium model constituted mainly of epithelial cells, an epidermis model constituted mainly of keratinocytes, a skin model constituted of an epidermis and of a dermis, a model of mucous membrane constituted of an epithelium and of a chorion, a model of biopsies (or explants) maintained in survival, as well as the models in a monolayer, or in suspension, making use of the cells present in the models described above.

Use in these models can be made of normal, healthy or pathological cells, or of cells which originate from cell-lines; these cells can be of human or animal origin.

According to a variant of this latter characteristic, the three-dimensional culture model of connective matrices (dermis or chorion), comprises a support sown with stromal cells in order to form reconstructed dermis or reconstructed chorions.

The three-dimensional epidermis or epithelium culture model comprises a support sown or not beforehand with stromal cells, in particular fibroblasts, and then with epithelial cells and in particular keratinocytes, so as to obtain reconstructed epithelia or epidermis.

The three-dimensional reconstructed skin or mucous membrane culture model comprises a matrix support (dermal or of chorion) sown with epithelial cells in order to obtain a reconstructed mucous membrane or with keratinocytes in order to obtain a reconstructed skin.

According to a variant, the three-dimensional culture model used comprises a model in which at least one additional cell type has been incorporated, e.g. endothelial cells (EC) and/or lymphocytes and/or adipose cells and/or skin appendices, such as body hair, hair, sebaceous glands.

According to a variant, the three-dimensional support can also enable the colonization by any other cell type (immune cells, endothelial cells, neurons, muscle cells, hepatocytes, etc. . . . ).

Advantageously, pigmentary cells, immunocompetent cells (Langerhans cells and/or dendritic cells), nerve cells . . . , can be introduced in addition to the epithelial part.

The different cell types (fibroblasts, keratinocytes, melanocytes . . . ) extracted are amplified separately and can be used separately or pooled from several donors for the reconstruction of the three-dimensional models as well as for the cultures in monolayers or in suspension.

The tissue models defined above are used at the end of the culture in order to make genomic and proteomic analyses, which enable in particular the selection, the identification and the characterization of potential targets for fighting against the effects of a stress.

The potential targets correspond to the biological parameters which are to be reversed or the modification of which is to be indicated.

After definition of the targets, these same models and methods of detection can be used for the screening of cosmetic or pharmaceutical active principles. These same models and methods of detection can be used for the demonstration of effectiveness of cosmetic or pharmaceutical formulations containing, or not, the actives.

These models and methods of detection can also be used for demonstrating the toxicity of actives, cosmetic or pharmaceutical, or of cosmetic or pharmaceutical formulations, this toxicity being induced by a stress, in particular by phototoxicity.

Amongst the analytical techniques used, the following can be cited in particular:

for the analysis of the proteomic profile: bidimensional electrophoresis, and/or protein arrays and/or cytokine array, and/or combined ELISA determinations,

for the analysis of the genomic profile: DNA Arrays, and/or polymerase chain reaction multiplex (PCR-multiplex), and/or polymerase chain reaction (PCR), and/or real time polymerase chain reaction (real time PCR),

for the analysis of the transcriptomic profile: RNA arrays, cDNA arrays and/or reverse transcription polymerase chain reaction multiplex (RT-PCR-multiplex) and/or reverse transcription polymerase chain reaction (RT-PCR) and/or real time reverse transcription polymerase chain reaction (real time RT-PCR).

### DETAILED DESCRIPTION OF THE INVENTION

According to a first aspect, the invention relates to a method of identifying an eventual modification of at least one biological parameter comprising the compared proteomic and/or compared transcriptomic and/or compared genomic analysis:

a) of living cells which are subjected to a stress, called stressed cells,

b) of living cells which are not subjected to this same stress, called reference cells,

c) at least one of these two classes of cells being used in a three-dimensional tissue model,

enabling eventually identifying at least one biological parameter which is modified following said stress.

Notably, the invention relates to a method of identifying an eventual modification of at least one biological parameter, comprising:

a) the proteomic analysis and/or transcriptomic analysis and/or genomic analysis, partial or complete, of living cells which are subjected to a stress, called stressed cells, sown in a tissue model;

b) comparing the analysis carried out in a) with the proteomic analysis and/or genomic analysis, partial or complete, of living cells which are not subjected to said stress, called reference cells; and

c) eventually identifying at least one biological parameter which is modified following said stress.

Advantageously, the reference cells and the stressed cells are used in a three-dimensional tissue model.

Advantageously, said biological parameter, which is modified during a stress, is defined by at least one difference between the metabolism of the cells called stressed cells and the metabolism of the cells called reference cells.

According to an advantageous embodiment, step a) cited above comprises the following steps:

a1) sowing one or more types of cells called reference cells in a cell model, i.e. in a monolayer, or in suspension, and/or tissue model, i.e. three-dimensional;

a2) exposing this model to a stress, said cells thus being designated stressed cells.

According to another advantageous embodiment, step a) comprises the following steps:

a1) exposing one or more types of living cells to a stress, the cells thus being called stressed cells:

a2) using these stressed cells, in a cell model, i.e. in a monolayer, or in suspension, and/or tissue model, i.e. three-dimensional;

Advantageously, the stress is a physical stress, this stress being selected from the stresses: UVA, UVB, sunlight, infra-red, near infra-red, thermal, magnetic field, hertzian radiation including microwaves and waves of mobile telephones, ionizing radiation including beta, gamma, X rays, such as those undergone during an accidental or non-accidental exposure to such radiation, and/or a physico-chemical stress, and/or biological stress, and/or mechanical stress.

According to an advantageous embodiment, the stressed cells are either:

cells which originate from biopsies taken in areas which are exposed to the sun, the person skilled in the art will know the area to be sampled, which will for example be the hand, the face.

cells which are stressed by a physical stress, this, stress being selected from the stresses: UVA, UVB, sunlight, infra-red, near infra-red, thermal, magnetic field, hertzian radiation including microwaves and waves of mobile telephones, ionizing radiation including beta, gamma, and X rays, such as those undergone during an accidental or non-accidental exposure to such radiation, and/or a physico-chemical stress, and/or biological stress, and/or mechanical stress.

According to an advantageous embodiment, the reference cells are either cells removed on biopsies which are not very stressed with solar radiation such as the breast, the abdomen, the foreskin, or cells which are non-stressed by a stress such as a physical stress of UVA and/or UVB and/or solar radiation type, and/or radiation from a magnetic field, and/or chemical stress, and/or biological stress and/or mechanical stress.

Advantageously, said stressed cells are cells from at least one human being or from at least one animal.

Advantageously, said study comprises at least one analysis selected from the following methods of analysis:

for the analysis of the proteomic profile: bidimensional electrophoresis, and/or protein arrays and/or cytokine array, and/or combined Elisa determinations,

for the analysis of the genomic profile: DNA arrays, and/or polymerase chain reaction multiplex (PCR-multiplex), and/or polymerase chain reaction (PCR), and/or real time polymerase chain reaction (real time PCR),

for the analysis of the transcriptomic profile: RNA arrays, cDNA arrays and/or reverse transcription polymerase chain reaction multiplex (RT-PCR-multiplex) and/or reverse transcription polymerase chain reaction (RT-PCR) and/or real time reverse transcription polymerase chain reaction (real time RT-PCR).

Advantageously, said tissue model is cultivated and/or preserved under conditions which maintain, at least partially, a cell metabolism.

Advantageously, said tissue model comprises at least fibroblasts or keratinocytes.

Advantageously, said model comprises:

normal, healthy or pathological cells, or cells which originate from cell-lines, preferably these cells are of human or animal origin.

Advantageously, said tissue model is selected from the following models:

a model of connective matrix, called dermis in the case of skin and called chorion in the case of a mucous membrane, containing mainly stromal cells, an epithelium model constituted mainly of epithelial cells, an epidermis model constituted mainly of keratinocytes, a skin model constituted of an epidermis and of a dermis, a mucous membrane model constituted of an epithelium and of a chorion.

Advantageously, said tissue model is a tissue model of connective matrix (dermis or chorion) comprising a matrix support preferably selected from:

an inert support selected from the group consisting of a semi-permeable synthetic membrane, in particular a semi-permeable nitrocellulose membrane, a semi-permeable nylon membrane, a teflon membrane or a teflon sponge, a semi-permeable membrane of polycarbonate or polyethylene, polypropylene, polyethylene terephthalate (PET), a semi-permeable Anopore inorganic membrane, of a cellulose acetate or cellulose ester (HATF) membrane, a semi-permeable Biopore-CM membrane, a semi-permeable polyester membrane, a membrane or a film of polyglycolic acid. In this group, the dermal models Skin ²™ model ZK1100 and Dermagraft® and Transcyte® (Advanced Tissue Sciences) for example, are found;

a cell culture-treated plastic (formation of a dermal leaf: Michel M. et al in In Vitro Cell. Dev Biol.-Animal (1999) 35: 318-326);

a gel or a membrane based on hyaluronic acid (Hyalograft® 3D—Fidia Advanced Biopolymers) and/or on collagen and/or on fibronectin and/or on fibrin; in this group, dermal model Vitrix® (Organogenesis) for example is found;

a porous matrix, which is surfaced or non-surfaced, made from collagen being able to contain one or more glycosaminoglycans and/or eventually chitosan (EP 0 296 078 A1 of the CNRS, WO 01/911821 and WO 01/92322 of Coletica); in this group, dermal model Mimederm® (Coletica) for example, is found, these matrix supports comprising stromal cells, in particular fibroblasts.

Advantageously, said tissue model is an epidermis tissue model or epithelium tissue model comprising a matrix support preferably selected from:

an inert support selected from the group consisting of a semi-permeable synthetic membrane, in particular a semi-permeable nitrocellulose membrane, a semi-permeable nylon membrane, a teflon membrane or a teflon sponge, a semi-permeable membrane of polycarbonate or polyethylene, polypropylene, of polyethylene terephthalate (PET), a semi-permeable Anopore inorganic membrane, of a cellulose acetate or cellulose ester (HATF) membrane, a semi-permeable Biopore-CM membrane, a semi-permeable polyester membrane; in this group, the following reconstructed models are found: Skinethic®, EpiDerm®, EpiAirway®, EpiOccular® (Mattek Corporation);

a film or a membrane based on hyaluronic acid and/or on collagen and/or on fibronectin and/or on fibrin. In this group, the models: Mimetop® (Coletica), Laserskin® (Fidia advanced Biopolymers), Episkin® (L'Oreal), in particular, can be cited.

These models are sown beforehand with stromal cells, in particular fibroblasts, and then with epithelial cells and in particular keratinocytes.

Advantageously, in the epithelial part, epithelial cells, pigmentary cells, immunocompetent cells, nerve cells, are introduced in addition to the epithelial cells, preferably, the immunocompetent cells are Langerhans cells.

Advantageously, said tissue model is a reconstructed skin or mucous membrane tissue model comprising a dermal or chorion matrix support preferably selected from:

an inert support selected from the group consisting of a semi-permeable synthetic membrane, in particular a semi-permeable nitrocellulose membrane, a semi-permeable nylon membrane, a teflon membrane or a teflon sponge, a semi-permeable membrane of polycarbonate or polyethylene, polypropylene, of polyethylene terephthalate (PET), a semi-permeable Anopore inorganic membrane, of a cellulose acetate or cellulose ester (HATF) membrane, a semi-permeable Biopore-CM membrane, a semi-permeable polyester membrane, said inert support containing stromal cells, in particular fibroblasts,

a gel based on collagen and/or hyaluronic acid and/or fibronectin, and/or on fibrin comprising stromal cells, in particular fibroblasts,

a porous matrix, which is surfaced or non-surfaced, made from collagen being able to contain one or more glycosaminoglycans and/or eventually chitosan, these porous matrices integrating stromal cells, in particular fibroblasts,

a de-epidermisised dermis or dead dermis, human or animal.

In this group, the models Mimeskine (Coletica), Apligraf® (Organogenesis), ATS-2000 (CellSystems® Biotechnologie Vertrieb), as well as Skin ²™ (ZK1200-1300-2000—Advanced Tissue Science), in particular, can be cited.

Moreover, there do exist models which are dedicated to tissue therapy which can also be the subject of such studies. The models Epidex™ (Modex Therapeutiques), Epibase® (Laboratoire Genevrier), Epicell™ (Genzyme), Autoderm™, and Transderm™ (Innogenetics), can be cited.

The matrix support is then sown with epithelial cells in order to obtain a reconstructed mucous membrane or with keratinocytes in order to obtain a reconstructed skin.

Advantageously, said tissue model used comprises a model in which at least one additional cell type has been incorporated, preferably endothelial cells (EC) and/or immune cells such as lymphocytes, macrophages, dendritic cells and/or adipose cells and/or skin appendices, such as body hair, hair, sebaceous glands.

According to a second aspect, the invention relates to the use of a method as defined above for carrying out the screening of at least one potentially active substance capable of reversing at least one biological parameter which is modified during a stress as defined above.

According to a third aspect, the invention relates to the use of a method for carrying out the screening of at least one potentially active substance capable of providing an indication of the modification of at least one biological parameter which is modified during a stress as defined above.

Advantageously, the present invention relates to a method for carrying out the screening of at least one potentially active substance capable of providing an indication of or of reversing the modification of at least one biological parameter which is modified during a stress as defined above, comprising:

A/ placing said potentially active substance in contact with reference cells as defined above, sown in a tissue model as defined above, for a period of time sufficient to enable said potentially active substance to act; applying a stress as defined above;

B/ proteomic analysis and/or transcriptomic analysis and/or genomic analysis, partial or complete, for making the study of the action of said substance on the cell metabolism of said cells;

C/ comparing the cell metabolism of said cells in the presence of the potentially active substance with the metabolism of said cells without the presence of said substance, called control cells, and;

D/ identifying the presence or the absence of activity of said potentially active substance, notably comprises identifying a positive or negative effect of said substance in order to provide an indication of the modification of the biological parameter.

According to another aspect, the invention relates to a method of identifying at least one potentially active substance capable of reversing at least one biological parameter which is modified during a stress comprising:

a) culturing cells which are subjected to a stress, called stressed cells preferably as defined above, having a modified biological parameter, in the presence of at least one eventually active substance, for a period of time sufficient to enable said potentially active substance to eventually act on the cell metabolism of said cells, said stressed cells being sown in a tissue model as defined above;

b) the proteomic analysis and/or transcriptomic analysis and/or genomic analysis, partial or complete, preferably as defined above, of the stressed cells which are cultivated in step a);

c) comparing the analysis carried out in b) with the proteomic analysis and/or transcriptomic analysis and/or genomic analysis, partial or complete, preferably as defined above, of living cells which are cultivated without the presence of said potentially active substance, called control cells;

d) following the comparison of the analyses carried out in c), eventually identifying at least one active substance capable of reversing at least one biological parameter which is modified by the stress.

According to another aspect, the invention relates to a method of identifying at least one potentially active substance capable of providing an indication of the modification of at least one biological parameter which is modified during a stress comprising:

a) placing said potentially active substance in contact with reference cells as defined above, sown in a tissue model as defined above, for a period of time sufficient to enable said potentially active substance to act; applying a stress as defined above;

b) the proteomic analysis and/or transcriptomic analysis and/or genomic analysis, partial or complete, preferably as defined above, of the stressed cells which are cultivated in step a);

c) comparing the analysis carried out in b) with the proteomic analysis and/or genomic analysis, partial or complete, preferably as defined above, of living cells which are cultivated without the presence of said potentially active substance, called control cells;

d) following the comparison of the analyses carried out in c), eventually identifying at least one active substance capable of providing an indication of the modification of at least one biological parameter which is modified by the stress.

According to another aspect, the invention relates to the use of an active substance selected by a method as defined above, for preparing at least one cosmetic and/or pharmaceutical composition.

According to another aspect, the invention relates to a substance which is active in the field of cosmetics or of pharmacy and which is selected by a method defined above.

According to another aspect, the invention relates to an active substance capable of reversing a biological parameter which is identified as being modified during a physical, chemical or biological stress, and/or of providing an indication of the modification thereof, this parameter having been identified by making compared studies made between cell models making use of cells called <<stressed>> cells and cell models making use of cells called <<reference>> cells, one at least of these models being a tissue model comprising at least either fibroblasts or keratinocytes.

Other aims, characteristics and advantages of the invention will appear clearly in the light of the explanatory description which follows and which is made in reference to the Examples which are given simply as an illustration and which in no way limit the scope of the invention. The Examples make up an integral part of the present invention, and any characteristic which appears novel with respect to any state of the art is claimed as an integral part of the invention in its function and in its generality. In the Examples, all percentages are given by weight, the temperature is given in degrees Celsius, the pressure is atmospheric pressure, unless indications to the contrary.

### EXAMPLE 1

Extraction and Culture of Cells Called <<Stressed Cells>> or of Cells Called <<Reference Cells>>

Cells which are Called <<Reference Cells>> are Either:

cells which are extracted from biopsies, from donors of varying age, which biopsies are non-stressed in the sun and which are obtained from plastic surgery preferably which is abdominal or mammary or eventually gingival or vaginal.

Cells which are Called <<Stressed Cells>> are either:

cells which are extracted from biopsies, from donors of varying age, which biopsies are obtained from plastic surgery from areas stressed in the sun (face, neck, hand).

The cell types obtained can be fibroblasts extracted by the technique of explants or par enzymatic digestion, e.g. with collagenase, keratinocytes or melanocytes extracted after enzymatic dermo-epidermic dissociation, in particular with dispase or thermolysin or trypsin-EDTA . . . .

After extraction, the fibroblasts are amplified in DMEM medium (Dulabecco's Modified Eagle's Medium)/Ham F12 glutamax 50/50 volume/volume, supplemented with 10% of calf serum, with penicillin at a final concentration of 100 IU/milliliter, with gentamycin at a final concentration of 1 microgram/milliliter, with amphotericin B at a final concentration of 1 microgram/milliliter. The fibroblasts are amplified by trypsination as soon as a confluence of 90% is obtained.

After extraction, the keratinocytes are amplified in K-SFM medium (Keratinocyte Serum Free Medium—Invitrogen) containing extract of bovine pituitary gland supplemented with penicillin at a final concentration of 100 IU/milliliter, with gentamycin at a final concentration of 1 microgram/milliliter, with amphotericin B at a final concentration of 1 microgram/milliliter. The keratinocytes are amplified by trypsination as soon as a confluence of 90% is obtained.

After extraction, the melanocytes are amplified in MMK2 medium (Melanocyte Medium Kit—Sigma) supplemented with penicillin at a final concentration of 100 IU/milliliter, with gentamycin at a final concentration of 1 microgram/milliliter, with amphotericin B at a final concentration of 1 microgram/milliliter and with geneticin at the rate of 100 micrograms/milliliter for 3 days so as to eliminate the residual keratinocytes. The culture is then continued in the same medium except the geneticin. The melanocytes are amplified by trypsination as soon as a confluence of 90% is obtained.

### EXAMPLE 2

Preparation of Reconstructed Dermis from Cells Called <<Stressed Cells>> or from Cells Called <<Reference Cells>>

500,000 fibroblasts which originate from a pool of three reference biopsies (mammary biopsy) and from a pool of three stressed biopsies (lifting), which are amplified as described in Example 1, are sown in dermal substrates made up of collagen which is cross-linked with diphenylphosphorylazide, in a DMEM-glutamax medium supplemented with 10% of calf serum, with ascorbic acid-2-phosphate at a final concentration of 1 millimolar, with EGF or epidermal growth factor at a final concentration of 10 nanogram/milliliter, with penicillin at a final concentration of 100 IU/milliliter, with gentamycin at a final concentration of 1 microgram/milliliter, with amphotericin B at a final concentration of 1 microgram/milliliter for a period of 21 days.

### EXAMPLE 3

Quantification of Cytokines in Reconstructed Dermis Sown Beforehand with Cells Called <<Stressed Cells>> or with Cells Called <<Reference Cells>>, the Stress Being an Irradiation with UVA

500,000 fibroblasts originating from a reference biopsy (mammary biopsy), which are amplified as described in Example 1, are sown in dermal substrates made up of collagen which is cross-linked with diphenylphosphorylazide, in a DMEM-glutamax medium supplemented with 10% of calf serum, with ascorbic acid-2-phosphate at a final concentration of 1 millimolar, with EGF or epidermal growth factor at a final concentration of 10 nanogram/milliliter, with penicillin at a final concentration of 100 IU/milliliter, with gentamycin at a final concentration of 1 microgram/milliliter, with amphotericin B at a final concentration of 1 microgram/milliliter, for a period of 15 days. They are then cultivated for a further week in medium without serum (FBM, Fibroblast Basal Medium—Promocell).

At the end of experimentation, the reconstructed dermis are rinsed in phosphate buffer (PBS) pH 7.4 and are then placed in small Petri dishes containing 1 ml of PBS at pH 7.4. Certain samples are preserved at ambient temperature (reconstructed dermis called “reference dermis”), and others are irradiated with UVA (365 nm) at increasing doses (0-5-10-15-20-25-30 J/cm²).

The reconstructed dermis comprising cells called <<stressed cells>> or <<reference cells>> are then incubated in a medium without serum (FBM, Promocell) for 24 hours. The cell viability in the matrices is evaluated by a test with MTT (methylthiazoletetrazolium) and is expressed in percentage of the non-irradiated control. The media are collected, centrifuged, and the cytokines content is determined by the Fluorokine MAP kit (R&D Systems). Briefly, 50 μl of standards or of samples are pipetted into identified wells. 50 μl of microparticles immobilizing specific antibodies of different cytokines are added into the wells as a function of a pre-defined plate plan. After one hour of incubation under orbital agitation, and elimination of the non-fixed substances by washing, labeled antibodies which are specific of different cytokines are added into the wells and are incubated for 2 hours under orbital agitation. After washing, the microparticles are suspended in washing buffer, are agitated for 1 minute under orbital agitation. The reading is carried out immediately on a Luminex 100 Analyser (R&D Systems). The contents of the different cytokines which are present in the media analyzed are determined by virtue of calibration ranges made with highly purified recombinant human cytokines. This technique enables an experimentation to analyze the cellular secretion of various cytokines (IL-1β, IL-2, IL-4, IL-6, IL-8, IL-10, TNFα, GM-CSF, G-CSF, VEGF, bFGF, G-CSF, IFNγ).

TABLE I

| | Non-stressed | Stressed cells |
|---|---|---|
| Cytokine (pg/ml) | cells | by UVA: 20 J/cm² |
| MTT | 100% | 72% |
| IL1 beta (pg/ml) | 24 ± 10 | 56 ± 19 |
| IL6 (pg/ml) | 570 ± 142 | 1210 ± 342 |
| IL8 (pg/ml) | 122 ± 17 | 173 ± 23 |
| TNF alpha (pg/ml) | 18 ± 6 | 110 ± 42 |

The methods of the invention enable seeing that the stress (a UVA irradiation, here), induces a decrease of the cell viability, as well as an increase of the synthesis of pro-inflammatory interleukins: it could thus be interesting to decrease these increases of synthesis by using active principles selected correctly.

### EXAMPLE 4

Quantification of Cytokines in Reconstructed Epidermis Comprising Cells Called <<Stressed Cells>> or Cells Called <<Reference Cells>>, for the Identification de Eventual Modulations of the Cytokines, the Stress Being for Example an Exposure to UVB Radiations

4.10⁶ reference keratinocytes, which here are non-exposed to UVB radiation (mammary biopsy), and which are amplified as described in Example 1 until passage 1 (first amplification by trypsination), are sown in Boyden chamber-type inserts (membrane of porosity 0.4 μm and diameter 25 mm), which are sown beforehand with a fibroblast nutrient under layers, in a DMEM-Glutamax/Ham F-12 (ratio 3/1 v/v) culture medium which is supplemented with 10% of Hyclone II calf serum, with ascorbic acid-2-phosphate at a final concentration of 1 millimolar, with EGF or epidermal growth factor at a final concentration of 10 ng/mL, with hydrocortisone at a final concentration of 0.4 micrograms/milliliter, with umulin at a final concentration of 0.12 IU/milliliter, with isuprel at a final concentration of 0.4 micrograms/milliliter, with triiodothyronine at a final concentration of 2.10⁻⁹ molar, with adenine at a final concentration of 24.3 micrograms/milliliter, with penicillin at a final concentration of 100 IU/milliliter, with amphotericin B at a final concentration of 1 microgram/milliliter, and with gentamycin at a final concentration of 20 micrograms/milliliter, and for a period of culture by immersion of 3 to 8 days.

The cultures of keratinocytes are then placed at the air-liquid interface for 12 to 18 days in the same culture medium as that used for the immersion culture, except the calf serum, the hydrocortisone, the isuprel, the triiodothyronine and the umulin.

6 samples undergo no treatment at the end of the culture (model comprising the reference cells=control) Different stresses can be made-on a whole of 6 reconstructed epidermis. For each type of stress, different protocols, selected from the following list, can for example be used:

Chemical stress: For 10 minutes to an hour, different agents are applied on the reconstructed epidermis and are then eliminated par rinsing in PBS, pH 7.4, such as, for example: 2% sodium lauryl sulfate (SLS), 0.05% tretinoine, hydrogen peroxide (3 to 300 μM), nitric oxide (NO) (MAHMA nonoate 0.1 to 1 μM), hypoxia by saturation with CO₂ or by cigarette smoke.

Biological stress: For one hour, various agents are applied on the reconstructed epidermis and are then eliminated par rinsing in PBS, pH 7.4, such as, for example: TGFβ (5-10 ng/ml), TNFα (50-100-200 IU/ml), IL1 (5-10 ng/ml), LPS (lipopolysaccharide 5-10 ng/ml).

Mechanical stress: the reconstructed epidermis are cut or compressed for 1 hour.

Thermal stress: the reconstructed epidermis are placed for one hour at 37° C., 40° C. and 43° C.

Magnetic field: the reconstructed epidermis are placed for one hour under a magnetic field, e.g. of 835 MHz/0.6W and 1,800 MHz/0.125W (radiofrequency of mobile telephones).

Microwaves: the reconstructed epidermis are subjected to microwaves frequencies 2.45 and 7.7 GHz and power 30 mW/cm².

Ionizing radiations: the reconstructed epidermis are subjected to doses of 0.2 to 10 mGy of X-Rays.

UV stress: UVA (0-60 J/cm²), UVB (0-100 mJ/cm²), sunlight (0-3,500 kJ/m²)

In this experimentation, we have chosen to carry out an irradiation of UVB type. For this, the culture-medium is eliminated and replaced with PBS pH 7.4 and certain reconstructed epidermis which are present in the inserts are irradiated at increasing doses of UVB (312 nm) from 0 to 100 mJ/cm². Others are preserved under the same conditions without irradiation (epidermis called “non-stressed reference epidermis”). The reconstructed epidermis thus treated are then incubated for 24 additional hours in an emersion medium. The determination of the cytokines is carried out by Florokine MAP as described in Example 3. The cell viability is evaluated by determination of proteins (bicinchoninic acid kit for protein determination—Sigma St Louis USA) or by another test of cell viability enabling the determination of the alkaline phosphatase enzymatic activity (incubation for 2 hours at 37° C. in a solution containing 5 mM of p-nitrophenyl phosphate, 0.1 M of sodium acetate, 0.1% of Triton X100 pH5 and then neutralization with 10% 1N NaOH and reading the absorbance at 405 nm).

The results are expressed, in percentage of the non-irradiated control, in the following Table:

TABLE II

| | Non-stressed | Stressed cells |
|---|---|---|
| Cytokine (pg/ml) | cells | by UVB: 21 mJ/cm² |
| BCA | 100% | 58% |
| IL1 beta (pg/ml) | 25 ± 8 | 137 ± 39 |
| IL6 (pg/ml) | 120 ± 30 | 702 ± 29 |
| IL8 (pg/ml) | 352 ± 57 | 473 ± 72 |
| TNF alpha (pg/ml) | 17 ± 11 | 125 ± 42 |

The methods of the invention enable seeing that the stress (here, a UVB irradiation), induces a significant decrease in the cell viability, as well as an increase in the synthesis of the pro-inflammatory interleukins: it could therefore be interesting to decrease these increases in synthesis and to limit the cell mortality by using active principles selected correctly.

### EXAMPLE 5

Preparation of Reconstructed Gingival Mucous Membrane Epithelia Comprising Cells Called <<Stressed Cells>> or Cells Called <<Reference Cells>>, Pour Quantification of the mRNAs of Cytokines, Chemokines and Immunomodulation Factors

1 to 2.10⁶ epithelial cells of gingival mucous membrane called <<reference gingival mucous membrane epithelia cells>> (gingival biopsy, Passage 3 (3^{rd} amplification by trypsination)), extracted as described in Example 1, are sown in Boyden chamber-type inserts (membrane of porosity 0.4 μm and diameter 10 mm—fibroblast nutrient under layer), in a DMEM-Glutamax/Ham F-12 (ratio 3/1 v/v) culture medium supplemented with 10% of Hyclone II calf serum, with ascorbic acid-2-phosphate at a final concentration of 1 millimolar, with EGF at a final concentration of 10 ng/mL, with hydrocortisone at a final concentration of 0.4 micrograms/milliliter, with umulin at a final concentration of 0.12 IU/milliliter, with isuprel at a final concentration of 0.4 micrograms/milliliter, with triiodothyronine at a final concentration of 2.10⁻⁹ molar, with adenine at a final concentration of 24.3 micrograms/milliliter, with penicillin at a final concentration of 100 IU/milliliter, with amphotericin B at a final concentration of 1 microgram/milliliter, and with gentamycin at a final concentration of 20 micrograms/milliliter, and for a period of culture by immersion of 3 to 8 days.

The cultures of epithelial cells are then maintained in immersion for 12 to 18 days in the same culture medium as that used for the immersion culture, except the percentage of calf serum which is lowered by 10% to 1%.

At the end of experimentation, the medium is eliminated and replaced with PBS pH 7.4, and the reconstructed epithelia Which are present in the inserts are stressed by various potentially irritant or sensitizing agents, at the rate of 20 μl per epithelium and for one hour: 5% sodium lauryl sulfate (SLS), Lipopolysaccharide (LPS) 1000 U/ml, an anti-inflammatory agent Prednisolone 10 mM (Sigma) and an active Inhipase® 3% (extract of the root of pueraria lobata, Coletica) also at the rate of 20 μl per epithelium, for 1 hour. The agents applied on the reconstructed epithelia are then eliminated and then the reconstructed epithelia are incubated for 24 additional hours in immersion medium without calf serum. Certain reconstructed epithelia are analyzed in terms of cell viability by a test with MTT (methylthiazoletetrazolium). Other reconstructed epithelia are scraped off and taken up into Tri Reagent® (T9424 Sigma, St Louis USA) and then extracted with chloroform. After centrifugation at 12,000 g for 15 minutes at 4° C., the RNAs are found in the upper layer.

The determination of the mRNAs of the epithelia is made by Expression Array (R&D System), according to the protocol defined by the supplier. The results are expressed as a factor of variation with respect to the non-stressed control: [(stressed cells results/non-stressed cells results)×100].

TABLE III

| | | Stress with | Effect of | Effect of |
|---|---|---|---|---|
| Labelers | Stress with SLS | LPSs | Prednisolone | Inhipase ® |
| PLA2 | 102 | 168 | 93 | 50 |
| IL1α | 241 | 131 | 62 | 52 |
| IL1β | 114 | 182 | 75 | 69 |
| IL-1ra | 82 | 75 | 112 | 98 |
| IL-1R | 102 | 124 | 95 | 119 |
| AcP | | | | |
| IL-1RI | 154 | 254 | 83 | 107 |
| IL-1RII | 137 | 262 | 88 | 102 |
| TNFα | 213 | 134 | 67 | 72 |
| IL6 | 163 | 342 | 81 | 135 |
| IL7 | 127 | 201 | 102 | 109 |
| IL8 | 183 | 287 | 105 | 92 |
| IL10 | 75 | 72 | 152 | 148 |
| IL11 | 112 | 125 | 101 | 108 |
| IL12 | 132 | 178 | 67 | 66 |
| IL15 | 147 | 189 | 99 | 108 |

The methods of the invention enable seeing that the stress (here, the application of an agent of irritant or sensitizing type), induced a modification of various labelers of the inflammation. The effectiveness of an active principle Inhipase® is demonstrated in comparison with the reference anti-inflammatory.

### EXAMPLE 6

Preparation of a Tissue Model of Reconstructed Skin and Study of a Thermal Stress on the Syntheses of mRNA and Comparison Between Young and Aged Donors.

The cells extracted are which are obtained from a photo-protected mammary biopsy.

400,000 fibroblasts called <<young fibroblasts>> (pool of three donors of less than 35 years old) and <<aged fibroblasts>> (pool of three donors of more than 55 years old) are extracted and amplified until passage 5 (5^{th} amplification by trypsination) as described in Example 1 and are then sown on the two faces of surfaced dermal substrates.

Briefly, the dermal substrates are prepared according to the following protocol:

Drying at 25° C. of a 0.75% collagen gel in order to form a film

Depositing the collagen film on a 0.75% collagen gel

Lyophilization for 24 h and cross-linking with DPPA (diphenylphosphorylazide 50 μl/g on collagen in dimethylformamide solvent and then pH 8.9 borate buffer)

After rinsing with demineralized water, the surfaced dermal substrates are lyophilized once again.

The medium used for the culture of the fibroblasts is a DMEM-Glutamax medium supplemented with 10% of Hyclone II calf serum, with ascorbic acid-2-phosphate at a final concentration of 1 millimolar, with EGF or Epidermal growth factor at a final concentration of 10 ng/mL, with penicillin at a final concentration of 100 IU/milliliter, with amphotericin B at a final concentration of 1 microgram/milliliter, and with gentamycin at a final concentration of 20 micrograms/milliliter. The obtaining of reconstructed dermis necessitates a period of culture of 14 days.

Then, 400,000 keratinocytes called <<young keratinocytes>> (pool of three donors of less than 35 years old) and <<aged keratinocytes>> (pool of three donors of more than 55 years old), which are extracted and amplified until passage 2 (2^{nd} amplification by trypsination) as described in Example 1, are sown on the surfaced dermal equivalents, on the film side, in a DMEM-Glutamax/Ham F-12 (ratio 3/1 v/v) culture medium supplemented with 10% of Hyclone II calf serum, with ascorbic acid-2-phosphate at a final concentration of 1 millimolar, with EGF at a final concentration of 10 ng/mL, with hydrocortisone at a final concentration of 0.4 micrograms/milliliter, with umulin at a final concentration of 0.12 IU/milliliter, with isuprel at a final concentration of 0.4 micrograms/milliliter, with triiodothyronine at a final concentration of 2.10⁻⁹ molar, with adenine at a final concentration of 24.3 micrograms/milliliter, with penicillin at a final concentration of 100 IU/milliliter, with amphotericin B at a final concentration of 1 microgram/milliliter, and with gentamycin at a final concentration of 20 micrograms/milliliter, and for a period of immersion culture of 7 days.

The cultures are then placed at the air-liquid interface for 14 days in the same culture medium as that used for the immersion culture, except the calf serum, the hydrocortisone, the isuprel, the triiodothyronine and the umulin.

At the end of experimentation, the medium is eliminated and replaced with PBS at pH 7.4, and the reconstructed skins which are present in the inserts are incubated for one hour at 37° C. (model comprising the reference cells) and one hour at 43° C. (model comprising the stressed cells). The reconstructed epidermis thus treated are then incubated for 24 additional hours in immersion medium. The samples comprising the “stressed cells” and the “reference cells” to heat shock are analyzed by cDNA array.

Briefly, the RNAs of the samples are extracted (eventually after grinding in liquid nitrogen with the aid of a biopulverizer) and purified according to the protocol of the supplier of Tri Reagent® (Sigma), for total elimination of the DNA.

the purified RNAs are analyzed qualitatively and quantitatively.

The following step was the purification of the pools of messenger RNAs (mRNAs) by hybridization of the poly(A) ends of the mRNAs with biotinylated oligo(dT) primers and selective capture on streptavidine beads, according to the protocol Atlas Pure (Clontech). The DNA probes which are multiply labeled with ³³P were made by reverse transcription of the mRNAs linked onto beads of poly(dT), with the aid of a pool of primers which are specific of the sequences which are immobilized on the <<arrays>>, in the presence of [α³³P]-dATP. The labeled probes were purified by exclusion column chromatography, the quality and the equivalence of the labeled probes were evaluated by liquid scintillation counting.

the Custom ATLAS membranes were pretreated and then the cDNAs which are immobilized on each membrane were hybridized (68° C., one night) with the corresponding labeled probes; the filters were then washed before analysis.

Analysis by autoradiography and quantification of the radioactivity of the spots with the aid of a Phosphorimager Cyclone (Packard instrument; 3 hours and then 72 hours of acquisition) and the QuantArray software, Packard.

Identification of the genes of interest varying between the different experimental conditions: young donors versus aged donors having or having not undergone a thermal stress. The results are expressed in percentage of variation between the aged model and young model, in non-stressed condition and stressed condition.

TABLE IV

| | | Aged/ |
|---|---|---|
| | Aged/Young | Young |
| Labelers | Non-stressed | Stressed |
| Phospholipase A2, proteine kinase C inhibitor | 101 | 136 |
| protein 1, factor activating exoenzyme S (FAS) | | |
| Bullous pemphigoid antigen 1 | 232 | 187 |
| Cartilage specific proteoglycan core protein | 53 | 82 |
| COL1A1 | Nd | 142 |
| COL1A2 | Nd | 124 |
| COL3A1 | 46 | 85 |
| COL6A1 | 64 | 62 |
| COL6A3 | 62 | 51 |
| Fibronectin precursor | 57 | 43 |
| Hyaluronan synthase | 189 | 154 |
| Involucrin | 260 | 159 |
| MMP1 | 68 | 125 |
| MMP11 | 63 | 92 |
| MMP16 | 135 | 195 |
| TIMP1 | 74 | 86 |
| TIMP3 | 173 | 152 |
| SPARC | 50 | 51 |
| Connective tissue growth factor precursor | 56 | 45 |
| Endothelin receptor type B | 51 | 47 |
| Glia derived neurite-promoting factor | 53 | 69 |
| Granulocyte chemotactic protein 2 | 562 | 489 |
| Growth inhibitory factor, metallothionein III | 161 | 142 |
| IL-1 alpha precursor | 162 | 398 |
| IL-1R1 | 54 | 64 |
| IL-1R2 | 512 | 242 |
| IL-1RA | 156 | 168 |
| IL-3 | 75 | 52 |
| IL-6 | 84 | 121 |
| IL-8 | 737 | 917 |
| IL-12B | 81 | 91 |
| KGF | 50 | 42 |
| Calgranulin B, migration inhibitory factor-related | 1981 | 1402 |
| protein 14, S100 calcium protein A9 | | |
| Migration inhibitory factor-related protein 8, | 4593 | 2567 |
| calgranulin A, S100 calcium protein A8, cystic | | |
| fibrosis antigen | | |
| Monocyte chemotactic protein 1, monocyte | 289 | 425 |
| chemotactic and activating factor, small | | |
| inductible cytokine A2 | | |
| Placenta growth factor 1 et 2 | 388 | 142 |
| Platelet derived growth factor A | 397 | 152 |
| Platelet derived growth factor receptor alpha | 50 | 89 |
| subunit | | |
| Pleiotrophin precursor, heparin binding factors | 49 | 58 |
| TGF beta inducible early protein | 48 | 102 |
| Prostaglandin G/H synthase 1 | 113 | 158 |
| Prostaglandin G/H synthase 2 | 68 | 125 |
| 47 kDa heat shock protein, collagen binding | 84 | 289 |
| protein 1 | | |
| 70 kDa heat shock protein | 131 | 587 |
| 90 kDa heat shock protein | 107 | 412 |
| Bax | 104 | 158 |
| bcl2 | nd | Nd |
| FAS antigen ligand (FASL) | nd | 289 |
| FAS antigen ligand receptor | nd | 452 |
| TNF-related apoptosis inducing ligand (TRAIL), | nd | 197 |
| apo2 ligand | | |
| Tenacin precursor, hexabrachion, cytotactin | 64 | 75 |

The methods of the invention enable seeing that the stress (here, a heat shock), induces, on the one hand, the modification of numerous labelers and on the other hand, a different response to the stress as a function of the age of the donors. This model thus enables defining action targets so as to provide an indication of or to reverse the effect of a heat shock. Furthermore, this model enables defining a different strategy for developing active principles as a function of the age group in question.

### EXAMPLE 7

Preparation of a Tissue Model of Pluricellular Reconstructed Skin Containing Langerhans Cells, Interstitial Dendritic Cells, Macrophages and Endothelial Cells, a Study of a Biological Stress, which is a Bacterial Aggression with Bacterial Lipopolysaccharide

Generation of the Undifferentiated and Immature Dendritic Cells which are Capable of Orientating Themselves Preferentially in the Pathway of Differentiation of the Langerhans Cells:

The peripheral circulating blood was collected taking a venous blood sample from one or more human donors, in vacutainers supplemented in usual anti-coagulant products such as lithium-heparin.

The separation of the monocytes (CD14⁺) from this circulating blood can be carried out advantageously according to the protocol described by Geissmann et al. in J. EXP. MED. Vol 187, No 6, 16 Mar. 1998, pages 961-966 published by The Rockefeller University Press in the following manner:

after centrifugation on a Ficoll® gradient (sodium diatrizoate/polysucchrose density 1.077 ; Lymphoprep Abcys 1053980), the mononucleated cells of the circulating blood are recovered and indirectly labeled with an antibody cocktail (mainly anti-CD3, anti-CD7, anti-CD19, anti-CD45RA, anti-CD56, anti-IgE) coupled to magnetic beads.

after passage on a magnetic column, only the non-magnetically-labeled monocytes are eluted.

The CD14⁺ monocytes are recovered in the eluate in proceeding by any physical method of separation well-known to the person skilled in the art, and notably by sedimentation or centrifugation, and are eluted as such for the subsequent cultures.

The CD14⁺ monocytes are then put into culture at the rate of about 1 million per milliliter, in an RPMI 1640 culture medium (Rosewell Park Memorial Institute) supplemented with 10% of foetal calf serum which is de-supplemented and initially containing two cytokines, namely the cytokine GM-CSF at the rate of 400 IU/mL and the cytokine TGFβ1 at the rate of 10 ng/mL.

The culture is done at 37° C. in a moist atmosphere containing 5% of CO₂.

The culture medium is initially supplemented with a third cytokine, namely the cytokine IL-13 at the rate of 10 ng/mL. Before at the most 2 days of culture, the same culture medium is added but not containing the IL-13 until the 6^{th} day of culture. On the 6th day, undifferentiated and immature dendritic cells are generated which are capable of orientating themselves preferentially in the pathway of differentiation into Langerhans cells:

about 60 to 80% of the dendritic cells which are generated in vitro express la Langerin at the intracellular level, and CCR6 which is the specific receptor of MIP-3α,

the dendritic cells which are generated in vitro are strongly chemo-attracted by MIP-3α, and this demonstrates the functionality of the receptor CCR6,

the dendritic cells which are generated in vitro are immature since they do not express the maturity labels CD83, DC-LAMP and CCR7.

The tissue model is then made according to the protocol:

2.10⁵ fibroblasts extracted from abdominal biopsy, called reference cells, are amplified, as is described in Example 1, and are then sown on dermal substrates based on collagen-glycosaminoglycan-chitosan, in a DMEM-Glutamax culture medium supplemented with 10% of hyclone II calf serum, with ascorbic acid-2-phosphate at a final concentration of 1 millimolar, with EGF or Epidermal growth factor at a final concentration of 10 ng/mL, with penicillin at a final concentration of 100 IU/milliliter, amphotericin B at a final concentration of 1 microgram/milliliter, and with gentamycin at a final concentration of 20 micrograms/milliliter, and for a period of culture of 21 days. The culture is continued for a further week in the medium described above except the EGF.

Then, 2.10⁵ keratinocytes extracted from abdominal biopsy, comprising cells which are called “reference cells” and amplified until the passage 1 (1^{st} amplification ) as described in Example 1, and 1 to 3.10⁵ undifferentiated dendritic cells which are generated in vitro, are sown on the dermal equivalents in a DMEM-Glutamax/Ham F-12 (ratio 3/1 v/v) culture medium supplemented with 10% of Hyclone II calf serum, with ascorbic acid-2-phosphate at a final concentration of 1 millimolar, with EGF at a final concentration of 10 ng/mL, with hydrocortisone at a final concentration of 0.4 micrograms/milliliter, with umulin at a final concentration of 0.12 IU/milliliter, with isuprel at a final concentration of 0.4 micrograms/milliliter, with triiodothyronine at a final concentration of 2.10⁻⁹ molar, with adenine at a final concentration of 24.3 micrograms/milliliter, with penicillin at a final concentration of 100 IU/milliliter, with amphotericin B at a final concentration of 1 microgram/milliliter, and with gentamycin at a final concentration of 20 micrograms/milliliter, and for a period of immersion culture of 7 days.

The cultures are then placed at the air-liquid interface for 20 days in the same culture medium as that used for the immersion culture, except the calf serum, the hydrocortisone, the isuprel, the triiodothyronine and the umulin.

In these conditions, the Langerhans cells are localized in the epidermis, the interstitial dendritic cells, the macrophages and the endothelial cells in the dermis.

The bacterial lipopolysaccharide (LPS). is added or not to the immersion medium at the rate of 10 ng/ml for 6 and 24 hours.

At the end of experimentation, the immunocompetent reconstructed skins are analyzed by cDNA array as is described in Example 6. The collected and frozen culture media are then analyzed by Florokine MAP as is described in Example 3. The results are presented in particular for the premature regulation part by interleukin 1 and TNFα in pg/ml and in percentage for the synthesis of cytokines ([(stressed cells results/non-stressed cells results)×100]) for the DNA array.

TABLE V

| | Absence | Stressed/ | Stressed/ |
|---|---|---|---|
| | of | reference | reference |
| Fluorokine MAP | stress | 6 hours | 24 hours |
| IL1 beta (pg/ml) | 76 ± 17 | 125 ± 28 | 207 ± 59 |
| TNF alpha (pg/ml) | 53 ± 21 | 87 ± 19 | 95 ± 22 |
| cDNA array | | | |
| interleukin-1 alpha (IL-1 alpha; | | 100 | 138 |
| IL1A); hematopoietin-1 | | | |
| interleukin-1 beta (IL-1; IL1B); | | 117 | 198 |
| catabolin | | | |
| interleukin-1 beta converting | | 100 | 125 |
| enzyme (IL1BCE) | | | |
| interleukin-1 receptor antagonist | | 72 | 335 |
| protein (IL-1RA; IRAP) | | | |
| interleukin-1 receptor type I (IL- | | 119 | 164 |
| 1R1); IL-1R-alpha; p80; CDW121A | | | |
| antigen | | | |
| interleukin-1 receptor type II (IL- | | 100 | 103 |
| 1R2); IL-1R-beta | | | |
| interleukin-1 receptor-associated | | 73 | 152 |
| kinase (IRAK) | | | |
| tumor necrosis factor alpha (TNF- | | 152 | 287 |
| alpha; TNFA); cachectin | | | |
| tumor necrosis factor alpha-induced | | 123 | 100 |
| protein 1, endothelial; B12 protein | | | |
| tumor necrosis factor receptor | | 83 | 119 |
| (TNFR) + tumor necrosis factor | | | |
| receptor 2 (TNFR2); tumor necrosis | | | |
| factor binding protein 2 (TBP2) | | | |
| tumor necrosis factor receptor 1 | | 108 | 176 |
| (TNFR1); tumor necrosis factor | | | |
| binding protein 1 (TBP1); CD120A | | | |
| antigen | | | |
| tumor necrosis factor-inducible | | 100 | 129 |
| protein TSG-6; hyaluronate-binding | | | |
| protein | | | |
| TNF-alpha converting enzyme | | 74 | 124 |
| (TACE); a disintegrin & | | | |
| metalloproteinase domain 17 | | | |
| (ADAM17) | | | |
| TNF-alpha-stimulated ABC protein | | 100 | 100 |
| (TSAP) | | | |
| TNF-related apoptosis inducing | | 96 | 98 |
| ligand (TRAIL); APO-2 ligand | | | |
| (APO2L) | | | |
| CD1a precursor | | 82 | 52 |
| CD86 antigen precursor | | 56 | 39 |
| CD40 antigen precursor | | 89 | 45 |

These results demonstrate a more or less rapid activation of the genes encoding the regulation of the inflammatory response by the interleukin 1 and the TNF alpha. The decrease observed in the case of the labelers CD1a, CD40 and CD86 is not due to a phenomenon of gene regulation but to a disappearance of the dendritic cells initially present in the three-dimensional models under the effect of the stress by the lipopolysaccharide, following their migration in the culture medium present under the insert.

The methods of the invention also enable making a selection of active principles which are able to provide an indication of or modulate the various modifications observed following the stress generated.

### EXAMPLE 8

Preparation of Pigmented Reconstructed Skins Exposed to a Stress Defined by a Repeated Solar Irradiation, and Study of the Effectiveness of Anti-Oxidant Active Principles

The cells extracted are obtained from a mammary biopsy non-exposed to the stress studied. 400,000 fibroblasts, amplified until passage 5 (5^{th} amplification by trypsination) as described in Example 1, are sown on dermal substrates based on surfaced sponges of collagen, in a DMEM-Glutamax culture medium supplemented with 10% of hyclone II calf serum, with ascorbic acid-2-phosphate at a final concentration of 1 millimolar, with EGF or Epidermal growth factor at a final concentration of 10 ng/mL, with penicillin at a final concentration of 100 IU/milliliter, with amphotericin B at a final concentration of 1 microgram/milliliter, and with gentamycin at a final concentration of 20 micrograms/milliliter, and for a period of culture of 14 days.

Then, 400,000 keratinocytes and 10,000 melanocytes, amplified until passage 2 (2^{nd} amplification by trypsination) as described in Example 1, are sown on the dermal equivalents in a DMEM-Glutamax/Ham F-12 (ratio 3/1 v/v) culture medium supplemented with 10% of Hyclone II calf serum, with ascorbic acid-2-phosphate at a final concentration of 1 millimolar, with EGF at a final concentration of 10 ng/mL, with hydrocortisone at a final concentration of 0.4 micrograms/milliliter, with umulin at a final concentration of 0.12 IU/milliliter, with isuprel at a final concentration of 0.4 micrograms/milliliter, with triiodothyronine at a final concentration of 2.10⁻⁹ molar, with adenine at a final concentration of 24.3 micrograms/milliliter, with penicillin at a final concentration of 100 IU/milliliter, with amphotericin B at a final concentration of 1 microgram/milliliter, and with gentamycin at a final concentration of 20 micrograms/milliliter, and for a period of immersion culture of 7 days.

The cultures are then placed at the air-liquid interface for 14 days in the same culture medium as that used for the immersion culture, except the calf serum, the hydrocortisone, the isuprel, the triiodothyronine and the umulin.

Twice per week and for two weeks, the immersion medium is eliminated and replaced by PBS at pH 7.4. Certain pigmented reconstructed skins present in the inserts are preserved at ambient temperature; this model comprises cells which are called “reference cells”. Other pigmented reconstructed skins present in the inserts are irradiated at 561 KJ/m² (corresponding to an average hour of exposure in Central Europe) with the aid of a solar irradiator Suntest CPS+ (ATLAS); this model comprises cells which are called “stressed cells”. Out of the periods of irradiation, the reconstructed skins are cultivated at 37° C. under 5% of CO₂ in immersion medium.

8 μl of a cosmetic formulation containing or not containing an anti-oxidant active at 3%, e.g. Flavagrum® (Hesperitine Laurate, Coletica), Flavenger® (Quercitine Caprylate, Coletica), are applied on the pigmented reconstructed skins for 10 days.

At the end of the treatment, the pigmented reconstructed skins are immersed for 24 additional hours in immersion medium, and then the effectiveness of the anti-oxidant treatment is evaluated by analysis of:

The cellular viability (test with MTT-methylthiazoletetrazolium) in the pigmented reconstructed skins comprising the “stressed” cells or the “reference” cells. The results are expressed in percentage of variation with respect to the non-irradiated control.

The secretion of interleukin quantified by Fluorokine MAP kit in the culture medium collected as described in Example 3. The results are expressed en picogram/ml.

TABLE VI

| | Non- | Stressed | | |
|---|---|---|---|---|
| | stressed | cells | Irradiation + | Irradiation + |
| | cells | (Irradiation) | Flavagrum ® | Flavenger ® |
| MTT | 100 | 76% | 88% | 92% |
| IL1 beta (pg/ml) | 76 | 179 | 125 | 97 |
| IL6 (pg/ml) | 379 | 649 | 452 | 426 |
| IL8 (pg/ml) | 275 | 395 | 312 | 294 |
| TNF alpha | 53 | 152 | 105 | 89 |
| (pg/ml) | | | | |

The methods of the invention enable seeing that the stress (solar irradiation here), induces a decrease in the cell viability, as well as an increase in the synthesis of pro-inflammatory interleukins: it is therefore interesting to limit the synthesis of pro-inflammatory molecules by using active principles selected correctly. Amongst the active principles screened, two of them, Flavagrum® and Flavenger®, have shown an effectiveness capable of tending to restoring the reference level for these two parameters.

### EXAMPLE 9

Study of a Physical Stress Defined by an Irradiation with UVB, on a Reconstructed Skin, and Study of Effectiveness of an Active Principle, the Analysis Being Carried out by Real Time RT-PCR

Reconstructed skins are made according to the protocol described in Example 6.

The UVB irradiation at the rate of 50 mJ/cm² is made for half the samples comprising <<stressed>> cells. The other samples are preserved at ambient temperature under the same conditions, and constitute samples comprising <<reference>> cells. The samples are then incubated for 24 additional hours in the presence or not of an active (1 and 3% of Flavenger®, i.e. acylated quercitine, Coletica, France).

At the end of experimentation, the content of tropoelastin mRNA, collagen of type I and collagen of type III is evaluated by a real time RT-PCR technique. For this, couples of primers enabling the amplification of specific fragments of tropoelastin, of collagen of type I and of collagen of type III (sense 18/antisense 19 and sense 18/antisense 20, respectively) and actin sequence primers (541 pairs of bases) were used. After extraction in TriReagent® (Sigma) and purification of the RNAs according to the protocol of the supplier, the reactions of RT-PCR (Reverse Polymerase Transcription Chain reactions) are carried out by quantitative real time RT-PCR with the aid of the <<Opticon>> system (MJ Research).

| | |
|---|---|
| COLL1 sense | CAGAGGGAAGCCGCAAGA |
| COLL1 antisense | CTGGCCGCCATACTCGAAC |
| COLL3 sense | AAGGAGAGCCCGGACCAC |
| COLL3 antisense | GGACCTCCAGGGACGCCATC |
| ELASTIN sense | CCTTCCCCGCAGTTACCTTTC |
| ELASTIN antisense | GCACGCCACCTGGGTATACAC |
| ACTIN sense | GTGGGGCGCCCCAGGCACCA |
| ACTIN antisense | CTCCTTAATGTCACGCACGATTTC |

The reaction mixture (50 μl) introduced into the well is the following, for each sample:

10 μl of RNA at the concentration of 5 ng/μl.

The primers of the various labelers used

Reaction mixture (Qiagen-25 μl 2×QuantiTect SYBR Green RT-PCR master mix containing 5 mM MgCl2+0.5 μl QuantiTect RT mix), the labeler SYBR Green I inserting itself in the DNA double strand during elongation step).

The Conditions of RT-PCR are the Following:

Reverse transcription: 30 min at 50° C.,

PCR reactions: [15 sec at 94° C., 30 sec at 60° C. and 30 sec at 72° C.], 50 cycles.

The absence of contamination and the purity of the products amplified are verified by the melting curves of the amplified PCR products. The products having a double peak or an abnormal melting temperature are eliminated.

Analysis and Method of Calculation:

The incorporation of fluorescence in the amplified DNA was evaluated continuously during the PCR cycles. This system enables obtaining fluorescence measurement curves as a function of the number of PCR cycles and thus to evaluate a relative quantity of amplified DNA.

In order to take account of the:cell population present in the reconstructed skins, all the results were attributed to the signal <<actin>>, used as housekeeping gene.

According to the experimentation, the measurement threshold of the C(T) (=cycle threshold) is fixed for T between 0.05 and 0.01 and then an arbitrary unit of measurement is calculated for each gene according to the formula:

Sgene <<x>>=10⁷X(½)^{C(T)gene <<X>>}

C(T)gene <<x>> signifying the measurement threshold of the C(T) (Cycle Threshold) of the gene <<x>>.

The values of the genes of interest are attributed to the signal <<actin>> by calculation of the ratio:

R=Sgene <<x>>/Sactin.

These ratios are compared between the treated and non-treated samples.

TABLE VII

| | | Stressed | Irradiated + | Irradiated + |
|---|---|---|---|---|
| | Non-stressed | cells | Flavenger ® | Flavenger ® |
| | cells | (Irradiated) | 1% | 3% |
| Collagen I | 0.86 | 1.08 | 0.94 | 0.92 |
| Collagen III | 1.20 | 1.47 | 1.37 | 1.26 |
| Elastin | 4.20 | 5.2 | 4.87 | 4.61 |

The methods of the invention enable seeing that the stress (a UVB irradiation here), induces a rapid increase in the RNAs encoding the synthesis of molecules of the extra-cellular matrix such as the collagen of type I, of type III and the elastin. The application of an active principle Flavenger® enables limiting the effects of the UVB stress induced by restoring, in a dose-dependent manner, the synthesis of these molecules.

### EXAMPLE 10

Study of a Physical Stress Defined by a Solar Irradiation on Reconstructed Dermis and Screening of Active Principles, the Analysis Being Carried Out by Real Time RT-PCR Multiplex

The reconstructed dermis are made as described in Example 3, over 3 weeks.

Certain reconstructed dermis are preserved at ambient temperature; this model comprises cells called “reference cells”. Other reconstructed dermis are irradiated at 561 KJ/m² (corresponding to an average hour of exposure in Central Europe) with the aid of a solar irradiator Suntest CPS+ (ATLAS); this model comprises cells which are called “stressed cells”.

The reconstructed dermis are irradiated in the presence or not of active (3%) and are then incubated for 24 hours.

The RNAs are finally extracted as described in Example 5.

The expression of the genes of the latent TGF and of the collagen of type I (COL1) is analyzed simultaneously by real time RT-PCR Multiplex after a rigorous selection of the primers (yield, specificity and multiplex compatibility) and optimization of the experimentation of real time RT-PCR (concentrations of the components, parameters of the cycles, conditions of fluorescence detection).

The probes of hydrolysis of actin (20 to 30 mer) are labeled at the 5′ end with the JOE fluorescent reporter (Excitation 520-Emission 548) and at the 3′ end by the TAMRA quencher (Applied Biosystems—Foster City, Calif.). The probes of hydrolysis of the genes to be analyzed (20 to 30 mer) are labeled at the 5′ end with the fluorescent reporter FAM (Excitation 495-Emission 520) and at the 3′ end by the TAMRA quencher (Excitation 555-Emission 576—Applied Biosystem).

| | |
|---|---|
| TGF latent sense | AGCGGGAGGAGGGACGAG |
| TGF latent | TGAGGGACGCCGTGTAGG |
| antisense | |
| COL1 sense | CAACATGGAGACTGGTGAGACCTGCGTGTA |
| COL1 antisense | CTTGTCCTTGGGGTTCTTGCTGATGTA |

The Conditions of RT-PCR are the Following:

Superscript kit one step RT-PCR with platinum Taq (Invitrogen)

ABI PRISM® 7000 Sequence Detection System (Applied Biosystems)

Reaction Mixture:

10 μl RNA at the concentration of 5 ng/μl

25 μl 2× reaction mix

2.5 μl primers, sense and antisense, 10 μM

1.8 μl MgSO4 50 mM

2 μl dNTP 5 mM

1 μl of the hydrolysis of each gene couple (actin/TGFl and actin/collagen I) 10 μM

1 μl RT/Taq mix

water qsp 50 μl

RT-PCR Protocol

RT 48° C. 30 min

Denaturation of the RT and activation of the polymerase 95° C. 5 minutes

50 CYCLES OF: 94° C. 15 sec −60° C. 30 sec −72° C. 30 sec

The analysis of the results (calculation of the ratio R=Sgene <<x>>/Sactin) is carried out as described in Example 10. The effect of the actives is analyzed in terms of potentiation of the activation of the latent TGF induced by solar irradiation of the actives as well as in terms of direct effect and/or rebound effect (via active TGF-β1 released) on the collagen of type I. The results of several interesting actives (Wheat extract, Soft Roe; Coletica) are presented in percentage variation with respect to the control which is non-irradiated and non-treated with active.

TABLE VIII

| | Latent TGF | COL 1 |
|---|---|---|
| Non-stressed cells | 100 | 100 |
| stressed cells (irradiated) | 124 | 107 |

The methods of the invention enable seeing that the stress (a UV irradiation, here), induces an increase in the synthesis of TGF beta and of collagen 1: it could therefore be interesting to mimic these increases in synthesis by using active principles selected correctly. Thus, here are the results obtained for the two extracts selected:

TABLE IX

| | TGF —latent | | Collagen I | |
|---|---|---|---|---|
| | Non-stressed | | Non-stressed | |
| | cells | Stressed cells | cells | Stressed cells |
| Extract of | 129 | 187 | 153 | 213 |
| wheat | | | | |
| Soft Roe | 111 | 154 | 99 | 147 |
| Extract | | | | |

### EXAMPLE 11

Use of Reconstructed Epidermis and of Cultures in Monolayer, Comprising <<Stressed>> Cells and <<Reference>> Cells for the Research of the Modulation of the Cutaneous Anti-Bacterial Capacities

Antibiotic peptides are molecules of small size (10 to 50 amino acids) which are capable of destroying microorganisms such as bacteria, fungi or viruses, by rendering their cell membrane permeable. The majority of the antibiotic peptides are found in the epithelial tissues of animals, where they play a preponderant role of first immune barrier. More particularly in man, they have been demonstrated in the gastro-intestinal and respiratory system, as well as in the skin and the mucous membranes. Defensins constitute a class of antimicrobial peptides which is the most studied. Two classes of defensins are distinguished, the α-defensins (6 representatives), and the β-defensins which are present in three forms, hBD1, hBD2 and hBD3 (Human β-defensin 1, 2 and 3).

Under a stress mimicking a microbial attack (lipopolysaccharide or LPS, TNF alpha, Interferon gamma, etc. . . . ), the cells can synthesize these molecules, as a means of defense.

In order to demonstrate this, keratinocyte cultures in monolayer and in the form of reconstructed epidermis are prepared from cells extracted from the same foreskin biopsy. The normal human keratinocytes are cultivated in monolayer on 96-wells culture plates, in a defined medium without serum and enriched with calcium (final concentration 1.7 mM).

At 80% of confluence, the cells are placed in contact with a chemical stress, i.e. molecules which mimic a microbial attack, such as TNFα (100 ng/ml) or IFNγ (100 ng/ml), for 16 hours. Keratinocytes which are non-stressed, i.e. which are not placed in contact with the chemical substances mimicking a microbial attack, are used in a model of reconstructed epidermis.

After 16 hours, the supernatants are collected and the cells are dry frozen at −80° C. after a rinsing with PBS.

The total RNAs are extracted with the aid of a 96-wells fluo extraction kit on silica columns and are determined on a 96-wells spectrophotometer at 260 and 280 nm. The RNAs are diluted at 5 ng/μl.

The one-step qualitative RT-PCR is carried out on 50 ng of RNA (initial) in 96 wells, on actin, hBD2 and hBD3. The primers are used at 0.5 μM and are from the literature: hBD2sense: 5′-CCAGCCATCAGCCATGAGGGT-3′; hBD2antisense: 5′-GGAGCCCJTlCTGMTC CGCA-3′ (Harder J. et al., A peptide antibiotic from human skin. Nature 1997; 387: 861); hBD3sense: 5′-AGCCTAGCAGCTATGAGGATC-3′, hBD3 antisense: 5′-CTTCGGCAGCATT TTCGGCCA-3′; actin sense: 5′-GTGGGGCGCCCCAGGCACCA-3′, actin antisense: 5′-CTCCTTMTGTCACGCACGAlTTC-3′ (Harder J. et al., Isolation and characterization of hBD3, a novel human inducible peptide antibiotic. J. Biol. Chem. 2001; 276: 5707-5713).

The samples are placed in a thermocycler and following a common program of amplification: 50° C., 30 mins; 94° C., 2 mins; (94° C., 30 secs; 60° C., 30 sec; 68° C., 30 sec) 32 cycles for the defensins and 30 cycles for the actin; 72° C., 10 min; 14° C., infinity.

After amplification, the products are mixed at the rate of 3 μl of products of amplification of actin+6 μl of products of amplification of hBD2+6 μl of products of amplification of hBD3. 5 μl of a mixture of filler buffer and water (2/3) are added and the final 20 μl are deposited on a pre-poured 2% agarose gel. The samples migrate in 30 minutes and the bands are visualized under UV in a black chamber and are photographed digitally.

2^{nd} Step of the Screening Method:

1 to 2.10⁶ foreskin keratinocytes, extracted as described in Example 1, are sown in Boyden chamber-type inserts (membrane of porosity 0.4 μm and diameter 10 mm), are sown beforehand with a fibroblast nutrient under layer, in a DMEM-Glutamax/Ham F-12 (ratio 3/1 v/v) culture medium supplemented with 10% of Hyclone II calf serum, with ascorbic acid-2-phosphate at a final concentration of 1 millimolar, with EGF at a final concentration of 10 nanograms/milliliter, with hydrocortisone at a final concentration of 0.4 micrograms/milliliter, with umulin at a final concentration of 0.12 IU/milliliter, with isuprel at a final concentration of 0.4 micrograms/milliliter, with triiodothyronine at a final concentration of 2.10⁻⁹ molar, with adenine at a final concentration of 24.3 micrograms/milliliter, with penicillin at a final concentration of 100 IU/milliliter, with amphotericin B at a final concentration of 1 microgram/milliliter, and with gentamycin at a final concentration of 20 micrograms/milliliter, and for a period of immersion culture of 3 days.

The cultures of keratinocytes are then placed at the air-liquid interface for 11 days in the same culture medium as that used for the immersion culture, except the calf serum, the hydrocortisone, the isuprel, the triiodothyronine and the umulin.

At the end of experimentation, the reconstructed epidermis are treated in the following manner:

1 sample undergoes no treatment (model comprising reference cells=negative control).

1 sample undergoes no treatment with the actives but undergoes various types of stress at the end of culture (model comprising stressed cells=positive control), e.g. incubation in the presence of TNFα at 100 ng/ml, IFN γ100 ng/ml.

1 sample undergoes the treatment with the active (1% in the culture medium for 24 hours) and then a stress (equivalent to the positive control).

The reconstructed epidermis are then incubated for 24 additional hours, in the presence or not of actives, are rinsed in PBS and then the total RNAs are extracted and determined on a 96-wells spectrophotometer at 260 and 280 nm. The RNAs are diluted at 5 ng/μl and are treated as described above.

Analysis:

The photographs of the gels are analyzed by an image treatment software which quantifies the intensity of the bands. The ratios of intensity of the hBD2/actin and hBD3/actin bands are compared on the one hand between the monolayer model and the 3D model (reconstructed epidermis) in a non-stressed condition, and, on the other hand, following the effect of a stress (cells treated with TNFβ or IFNγ).

Expression of hBD2 and hBD3 in Monolayer, Compared to a 3D Reconstructed Epidermis Model, Models Using Non-Stressed Reference Cells:

TABLE X

| | HBD2 | HBD3 |
|---|---|---|
| Non-stressed cells in monolayer | 0.318 | 0 |
| Non-stressed cells in 3D reconstructed epidermis | 0.525 | 0.015 |

Effects of the Stress on the Expression of hBD2 and hBD3 in Monolayer, Compared to a 3D Reconstructed Epidermis Model:

TABLE XI

| | HBD2 | HBD3 |
|---|---|---|
| Stressed cells by TNFα, in monolayer | 1.240 | 0.266 |
| Stressed cells by TNFα, in reconstructed epidermis | 1.617 | 0.546 |
| Stressed cells by IFNγ, in monolayer | 0.450 | 0.370 |
| Stressed cells by IFNγ, in reconstructed epidermis | 0.581 | 0.492 |

The response of a synthesis of defensins by the cells of the skin, during a stress mimicking a microbial attack is a defense response, that can therefore be sought to mimic in seeking active principles which are capable of stimulating the skin defensins without chemical aggression taking place.

What is claimed is:

1. A method of identifying an eventual modification of at least one biological parameter of living cells which comprises the steps of:
a) subjecting said living cells to a given stress, called stressed cells,
b) not subjecting said living cells to this given stress, called reference cells,
c) using at least one between the stressed cells and the reference cells in a three-dimensional tissue model,
d) performing at least one compared analysis of said stressed cells with said reference cells, said compared analysis being selected from the group consisting of a compared proteomic profile analysis, a compared transcriptomic profile analysis, and a compared genomic profile analysis; and
e) identifying whether at least one biological parameter of said living cells is eventually modified in said stressed cells.

2. The method of claim 1, wherein the reference cells and the stressed cells are used in a three-dimensional tissue model.

3. The method of claim 1, wherein said biological parameter, which is modified during said stress, is at least one difference between the metabolism of the stressed cells and the metabolism of the reference cells.

4. The method of claim 1, wherein said step a) comprises the following steps:
a1) sowing at least one type of the reference cells in at least one cell model selected from the group consisting of a monolayer, a suspension, and a three-dimensional tissue model;
a2) exposing this model to a stress, said cells thus being designated stressed cells.

5. The method of claim 1, wherein step a) comprises the following steps:
a1) exposing at least one type of said living cells to a stress, the cells so stressed being called stressed cells:
a2) using these stressed cells, in at least one cell model selected from the group consisting of a monolayer, a suspension, and a three-dimensional tissue model.

6. The method of claim 1, wherein the stress is at least one stress selected from the group consisting of: a physical stress, a physico-chemical stress, a biological stress, and a mechanical stress.

7. The method of claim 6, wherein said physical stress is selected from the group consisting of: UVA, UVB, sunlight, infra-red, near infra-red, thermal, magnetic field, hertzian radiation, microwaves, waves of mobile telephones, ionizing radiation, beta rays, gamma rays, and X rays.

8. The method of claim 1, wherein the stressed cells are selected from the group consisting of:
cells which originate from biopsies taken in areas which are exposed to the sun; and
cells which are stressed by a stress selected from the group consisting of: a physical stress, a physico-chemical stress, a biological stress, and a mechanical stress.

9. The method of claim 8, wherein said cells originating from biopsies taken in areas which are exposed to the sun are cells taken from an area selected from the group consisting of the hand, and the face.

10. The method of claim 8, wherein said physical stress is a stress selected from the group consisting of: UVA, UVB, sunlight, infra-red, near infra-red, thermal, magnetic field, hertzian radiation, microwaves, waves of mobile telephones, ionizing radiation, beta rays, gamma rays, and X rays.

11. The method of claim 1, wherein the reference cells are cells selected from the group consisting of cells originating from biopsies which are located in skin areas not permanently stressed with solar radiation, and cells which are non-stressed by a stress.

12. The method of claim 11, wherein said biopsies, in skin areas not permanently stressed with solar radiation, are from an area selected from the group consisting of: the breast, the abdomen, and the foreskin.

13. The method of claim 11, wherein the reference cells are non-stressed by a stress, said stress being at least one selected from the group consisting of: a physical stress of UVA, a physical stress of UVB, a physical stress of solar radiation type, a physical stress of radiation from a magnetic field, a chemical stress, a biological stress, and a mechanical stress.

14. The method of claim 1, wherein said living cells are cells from at least one mammal selected from the group consisting of: a human being, and an animal.

15. The method of claim 1, wherein for the analysis of the proteomic profile, the analysis is at least one selected from the group consisting of: a bidimensional electrophoresis, a protein array, a cytokine array, and a combined ELISA determination.

16. The method of claim 1, wherein for the analysis of the genomic profile, the analysis is at least one selected from the group consisting of: a DNA array, a polymerase chain reaction multiplex (PCR-multiplex), a polymerase chain reaction (PCR), and a real time polymerase chain reaction (real time PCR).

17. The method of claim 1, wherein for the analysis of the transcriptomic profile, the analysis is at least one selected from the group consisting of: an RNA array, a cDNA array, a reverse transcription polymerase chain reaction multiplex (RT-PCR-multiplex), a reverse transcription polymerase chain reaction (RT-PCR), and a real time reverse transcription polymerase chain reaction (real time RT-PCR).

18. The method of claim 1, wherein said tissue model is cultivated under conditions which maintain, at least-partially, a cell metabolism.

19. The method of claim 1, wherein said tissue model is preserved under conditions which maintain, at least partially, a cell metabolism.

20. The method of claim 1, wherein said tissue model is cultivated and preserved under conditions which maintain, at least partially, a cell metabolism.

21. The method of claim 1, wherein said tissue model comprises at least fibroblasts.

22. The method of claim 1, wherein said tissue model comprises at least keratinocytes.

23. The method of claim 1, wherein said model comprises living cells selected from the group consisting of: normal cells, healthy cells, pathological cells, and cells which originate from cell-lines.

24. The method of claim 1, wherein said tissue model is selected from the group of tissue models consisting of: a model of connective matrix, called dermis in the case of skin and called chorion in the case of a mucous membrane, containing mainly stromal cells, an epithelium model constituted mainly of epithelial cells, an epidermis model constituted mainly of keratinocytes, a skin model constituted of an epidermis and of a dermis, and a mucous membrane model constituted of an epithelium and of a chorion.

25. The method of claim 1, wherein said tissue model of connective matrix comprises a matrix support.

26. The method of claim 25, wherein said matrix support is selected from the group consisting of: an inert support provided with stromal cells, a gel or a membrane comprising stromal cells, and a porous matrix comprising stromal cells.

27. The method of claim 26, wherein said stromal cells are fibroblasts.

28. The method of claim 26, wherein said inert support is selected from the group consisting of: a plastic, and a semi-permeable synthetic membrane.

29. The method of claim 28, wherein said semi-permeable synthetic membrane is selected from the group consisting of: a semi-permeable nitrocellulose membrane, a semi-permeable nylon membrane, a teflon membrane, a teflon sponge, a semi-permeable membrane of polycarbonate, a semi-permeable membrane of polyethylene, a semipermeable membrane of polypropylene, a semi-permeable membrane of polyethylene terephthalate (PET), a semi-permeable Anopore™ inorganic membrane, a cellulose acetate membrane, a cellulose ester (HATF) membrane, a semi-permeable Biopore-CM™ membrane, and a semi-permeable polyester membrane.

30. The method of claim 26, wherein said gel or membrane is based on a component selected from the group consisting of: hyaluronic acid, collagen, fibronectin and fibrin.

31. The method of claim 26, wherein said porous matrix is made from a component selected from the group consisting of: collagen containing at least one glycosaminoglycan, collagen containing chitosan and collagen containing at least one glycosaminoglycan and chitosan.

32. The method of claim 1, wherein said tissue model is a model selected from the group consisting of: an epidermis tissue model, and an epithelium tissue model, comprising a matrix support.

33. The method of claim 31, wherein said matrix support is selected from the group consisting of: an inert support sown beforehand with stromal cells and then with epithelial cells, an inert support not sown beforehand with stromal cells but with epithelial cells, and a film or a membrane sown beforehand with stromal cells and then with epithelial cells.

34. The method of claim 33, wherein said stromal cells are fibroblasts.

35. The method of claim 33, wherein said epithelial cells are keratinocytes.

36. The method of claim 33, wherein said inert support is a semi-permeable synthetic membrane.

37. The method of claim 36, wherein said semi-permeable synthetic membrane is selected from the group consisting of: a semi-permeable nitrocellulose membrane, a semi-permeable nylon membrane, a teflon membrane, a teflon sponge, a semi-permeable membrane of polycarbonate, a semi-permeable membrane of -polyethylene, a semi-permeable membrane of polypropylene, a semi-permeable membrane of polyethylene terephthalate (PET), a semi-permeable Anopore™ inorganic membrane, a cellulose acetate membrane, a cellulose ester (HATF) membrane, a semi-permeable Biopore-CM™ membrane, and a semi-permeable polyester membrane.

38. The method of claim 33, wherein said film or membrane is based on at least one component selected from the group consisting of: hyaluronic acid, collagen, fibronectin, fibrin and mixtures thereof.

39. The method of claim 33, wherein in the epithelial part, additional cells are introduced.

40. The method of claim 39, wherein said additional cells are selected from the group consisting of: epithelial cells, pigmentary cells, immunocompetent cells, and nerve cells.

41. The method of claim 40, wherein said immunocompetent cells are Langerhans cells.

42. The method of claim 1, wherein said tissue model is selected from the group consisting of: a reconstructed skin model comprising a dermal or chorion matrix support, and a reconstructed mucous membrane tissue model comprising a dermal or chorion matrix support.

43. The method of claim 42, wherein said dermal or chorion matrix support is selected from the group consisting of: an inert support containing stromal cells, a gel comprising stromal cells, a porous matrix, and a de-epidermisized dermis or dead dermis.

44. The method of claim 43, wherein said stromal cells are fibroblasts.

45. The method of claim 43, wherein said inert support is a semi-permeable synthetic membrane.

46. The method of claim 45, wherein said semi-permeable synthetic membrane is selected from the group consisting of: a semi-permeable nitrocellulose membrane, a semi-permeable nylon membrane, a teflon membrane, a teflon sponge, a semi-permeable membrane of polycarbonate, a semi-permeable membrane of polyethylene, a semi-permeable membrane of polypropylene, a semi-permeable membrane of polyethylene terephthalate (PET), a semi-permeable Anopore inorganic membrane, a cellulose acetate membrane, a cellulose ester (HATF) membrane, a semi-permeable Biopore-CM membrane, a semi-permeable polyester membrane.

47. The method of claim 43, wherein said gel is based on at least one component selected from the group consisting of: collagen, hyaluronic acid, fibronectin, fibrin and mixtures thereof.

48. The method of claim 43, wherein said porous matrix is made from at least one component selected from the group consisting of: collagen containing at least one glycosaminoglycan, and chitosan.

49. The method of claim 43, wherein said dermis is of a dermis taken from a human or from an animal.

50. The method of claim 43, wherein said matrix support is then sown with epithelial cells in order to obtain a reconstructed mucous membrane.

51. The method of claim 43, wherein said matrix support is then sown with keratinocytes in order to obtain a reconstructed skin.

52. The method of claim 1, wherein said tissue model used comprises a model in which at least one additional cell type has been incorporated.

53. The method of claim 52, wherein said additional cell type that has been incorporated is at least one additional cell type selected from the group consisting of: endothelial cells (EC), immune cells, dendritic cells, adipose cells, and skin appendices.

54. The method of claim 53, wherein said immune cells are selected from the group consisting of: lymphocytes, and macrophages.

55. The method of claim 53, wherein said skin appendices are selected from the group consisting of: body hair, hair, and sebaceous glands.

56. A method for carrying out the screening of at least one potentially active substance capable of providing an effect selected from the group consisting of: an effect of reversing at least one biological parameter which is modified during a stress as defined in claim 1, and an effect of providing an indication of the modification of at least one biological parameter which is modified during a stress as defined in claim 1.

57. The method of claim 56 which comprises:
A/placing said potentially active substance in contact with reference cells as defined in claim 1, sown in a tissue model as defined in claim 1, for a period of time sufficient to enable said potentially active substance to act; applying a stress as defined in claim 6;
B/carrying out at least one analysis, partial or complete, selected from the group consisting of: a proteomic analysis, a transcriptomic analysis, and a genomic analysis, for making the study of the action of said substance on the cell metabolism of said cells;
C/ comparing the cell metabolism of said cells in the presence of the potentially active substance with the metabolism of said cells without the presence of said substance, called control cells, and;
D/identifying the presence or the absence of activity of said potentially active substance, in order to provide an indication of the modification of the biological parameter.

58. The method of claim 57, wherein said identification of the presence or absence of activity of said potentially active substance comprises identifying a positive or negative effect of said substance.

59. A method of identifying at least one potentially active substance capable of reversing at least one biological parameter which is modified during a stress comprising:
a) culturing cells which are subjected to a stress, called stressed cells, having a modified biological parameter, in the presence of at least one potentially active substance, for a period of time sufficient to enable said potentially active substance to potentially act on the cell metabolism of said cells, said stressed cells being sown in a tissue model as defined in claim 1;
b) at least one analysis, partial or complete, selected from the group consisting of: a proteomic analysis, a transcriptomic analysis, and a genomic analysis, of the stressed cells which are cultivated in step a);
c) comparing the analysis carried out in b) with the proteomic analysis and/or genomic analysis, partial or complete, of living cells which are cultivated without the presence of said potentially active substance, called control cells.

60. The method of claim 59, wherein said method comprises an additional step d):
d) identifying at least one active substance capable of reversing at least one biological parameter which is modified by the stress.

61. A method of identifying at least one potentially active substance capable of providing an indication of the modification of at least one biological parameter which is modified during a stress comprising:
a) placing said potentially active substance in contact with reference cells as defined in claim 1 sown in a tissue model as defined in claim 1, for a period of time sufficient to enable said potentially active substance to act; application of a stress as defined in claim 6;
b) at least one analysis selected from the group consisting of: a proteomic analysis, a transcriptomic analysis, and a genomic analysis, of the stressed cells which are cultivated in step a);
c) comparing the analysis carried out in b) with the proteomic analysis and/or transcriptomic analysis and/or genomic analysis, of living cells which are cultivated without the presence of said potentially active substance, called control cells.

62. The method of claim 59, wherein said method comprises an additional step d):
d) identifying at least one active substance capable of providing an indication of the modification of at least one biological parameter which is modified by the stress.

63. A method of preparing at least one composition selected from the group consisting of a cosmetic composition, and a pharmaceutical composition, comprising incorporating an active substance screened by the method of claim 56.

64. A substance which is active in the field of cosmetics and which is screened by the method of claim 56.

65. A substance which is active in the field of pharmacy and which is screened by the method of claim 56.

66. An active substance providing at least one effect selected from the group consisting of: an effect of reversing a biological parameter which is identified as being modified during a stress, and an effect of providing an indication of the modification thereof, this parameter having been identified by making compared studies made between cell models making use of <<stressed>> cells and cell models making use of <<reference>> cells, one at least of these models being a tissue model comprising at least either fibroblasts or keratinocytes.

67. The active substance of claim 66, wherein said stress is selected from the group consisting of: a physical stress, a chemical stress, and a biological stress.
